(19) United States
(12) Patent Application Publication
(10) Pub. No.: US 2012/0178995 A1
(43) Pub. Date: Jul. 12, 2012

(54) Method and Apparatus for Cleaning the Field of View of an Endoscopic Lens

(75) Inventor: John P. Newton, IV, Pittsburgh, PA (US)

(73) Assignee: Parametric Mechanisms, LLC, Pittsburgh, PA (US)

(21) Appl. No.: 13/344,242

(22) Filed: Jan. 5, 2012

Related U.S. Application Data
(60) Provisional application No. 61/429,876, filed on Jan. 5, 2011.
(60) Provisional application No. 61/448,737, filed on Mar. 3, 2011.

(51) Int. Cl. A61B 1/00

(52) U.S. Cl. 600/121

(57) ABSTRACT

An endoscope lens protection apparatus may include a clear indexable film strip positioned over the endoscope lens having an underside surface and an opposite exposed surface, such that the underside surface is positioned to face the endoscope lens, and the incision side surface is exposed; wherein the indexable film is movable from a clean film position to a used film position, such that, in operation, the indexable film is advanced from a point in front of the lens to a used film position out of the endoscope field of view, and may further include a sheath having a proximal end and a distal end defining a chamber therebetween configured to receive the endoscope body, an interior sheath surface defining a film supply channel and a film retraction channel. A sealing mechanism associated with the sheath may also be included.

### CROSS-REFERENCE TO RELATED APPLICATIONS

This application claims the benefit of U.S. Provisional Application Nos. 61/429,876, filed Jan. 5, 2011, entitled “Method and Apparatus for Cleaning the Field of View of Endoscopic Lens”, and 61/448,737, filed Mar. 3, 2011, entitled “Air Seal to Protect the Field of View of an Endoscope”, the entire contents of both of which are hereby incorporated by reference in their entirety.

### BACKGROUND OF THE INVENTION

1. Field of the Invention

The present invention relates to methods, systems, and apparatuses of maintaining a clear field of view through an endoscope during a minimally invasive surgery, and, in particular, to any rigid variety of endoscope in which the distal end of the scope resides inside a bodily cavity, and in which the proximal end is either jigged or directly manipulated by an operator outside of such cavity.

2. Description of Related Art

A common nuisance during the course of a minimally invasive surgery in human and/or animal surgery is the soiling of the endoscope lens with blood, humor, or debris. When this occurs, the image quality often degrades to a point where the surgeon must consider momentarily suspending the operation at hand, removing the endoscope from the bodily cavity, and cleaning the endoscope tip. These interruptions and distractions hamper the efficiency and quality of the surgery.

Often, in related prior art, attempts to clear the view of the endoscope while it is still protruding into the bodily cavity share a common similarity. In these cases, the lens itself is soiled and becomes the object of direct attention. Such cases involve the use of jetted saline sprays, various forms of mechanical wiping action, or a combination of such techniques.

Therefore, a need exists for apparatuses, systems and methods that refresh the image quality in a seamless way so that these nuisances are eliminated.

### SUMMARY OF THE INVENTION

An endoscope lens protection apparatus for an endoscope having an endoscope body having a proximal end and a distal end and a lens positioned at a distal end of the endoscope body may include an indexable film strip. The film strip may be positioned over the endoscope lens having an underside surface and an opposite exposed surface, such that the underside surface is positioned to face the endoscope lens, and the incision side surface is exposed. The film strip is movable from a clean film position to a used film position, such that, in operation, the indexable film is advanced from a point in front of the lens to a used film position out of the endoscope field of view. In one embodiment, a sheath could also be included. The sheath could have a proximal end and a distal end defining a chamber therebetween configured to receive the endoscope body, wherein an interior sheath surface defines a film supply channel and a film retraction channel. The supply channel and retraction channel may extend from a proximal end to a distal end of the sheath, wherein the indexable film extends and is movable along the length of the film supply channel, around a point in front of the endoscope lens, and along the length of the film retraction channel. The apparatus may also include at least one clean film reservoir and at least one used film reservoir associated with the sheath. The clean film reservoir could be a spool and the used film reservoir could be a spool. In some embodiments, a housing member defining an interior chamber could be positioned at the proximal end of the sheath, wherein the clean spool and used spool could be positioned therein. A sealing mechanism could also be associated with the sheath, where the sealing mechanism could be configured to seal the underside surface of the film from debris. The sealing mechanism could include a gas-seal system, which could be configured to supply a flow of gas propagating through the sheath chamber between the interior surface of the sheath and the endoscope body from the proximal end of sheath to the distal end of the sheath and between the endoscope lens and the film. The gas-seal system may include a compressed air supply, wherein the flow of gas is supplied from the compressed air supply. In similar embodiments, the apparatus could include a housing member defining an interior chamber for receiving at least one clean film reservoir and at least one used film reservoir, the housing member interior chamber being in fluid communication with the compressed air supply and the sheath chamber. Additionally, the sheath could include a film guide surface positioned at the distal end of the sheath, which also, may define a film supply aperture and film retraction aperture and at least one detent positioned directly adjacent in communication with the film supply aperture. The guide surface may define a plurality of groves extending from the at least one detent to the film guide surface. In yet another embodiment, the sealing mechanism may include a sealing boot including a boot body having a proximal end and a distal end and defining a hollow internal cavity having a proximal boot opening and a distal boot opening, wherein the boot body is positioned over the sheath, such that the distal end of the sheath and endoscope body are inserted into the boot internal cavity through the proximal boot opening, the distal boot opening including a perimetric seal configured to seal the film underside surface from debris.

Another aspect includes a method of maintaining a clear field of view during endoscopic surgery using an endoscope including an endoscope body having a proximal end and a distal end and a lens positioned at a distal end of the endoscope body, the method including the steps of: positioning the endoscope into a surgical incision of a patient; advancing an indexable film in front of the endoscope lens field of view; viewing the surgery site through the endoscope lens and the indexable film; and advancing the indexable film, when the endoscope field of view becomes obstructed, wherein the film positioned in the field of view is moved to a used film position, and an unused portion of the film in the endoscope field of view providing an unobstructed view of the surgery site through the endoscope lens. Advancing the indexable film may include advancing the film through a sheath positioned around the endoscope body. The method may also include sealing an underside surface of the film. Sealing could include providing a gas-flow through the sheath between an interior surface of the sheath and the endoscope body from the proximal end of the endoscope to the distal end of the endoscope and between the endoscope lens and the film, or, in an alternative embodiment, positioning a sealing boot defining a distal opening having a perimetric seal around the distal end of the sheath.

In yet another aspect, an endoscope system could include: an endoscope body having a proximal end and a distal end and a lens positioned at a distal end of the endoscope body; an indexable film strip; and a sheath having a proximal end and a distal end defining a chamber therebetween configured to receive the endoscope body, an interior surface of the sheath defining a film supply channel and an opposite film retraction channel, the supply channel and retraction channel extending from the proximal end to the distal end of the sheath, wherein the indexable film extends from and is movable from the proximal end of the sheath, through the film supply channel, around a point in front of the endoscope lens in the endoscope field view, and through a length of the film retraction channel back to the proximal end of the sheath.

### BRIEF DESCRIPTION OF THE DRAWINGS

FIG. 1 is a perspective view of an endoscope according to the prior art;

FIG. 1A is an enlarged view of a distal end of the endoscope of FIG. 1 taken at line I-I;

FIG. 1B is a perspective view of a distal end of an alternative embodiment of an endoscope;

FIG. 2 is a perspective view of an embodiment of an endoscope lens protection apparatus;

FIG. 2A is a cross-section of the endoscope lens protection apparatus taken at line II-II of FIG. 2;

FIG. 2B is an enlarged view of a distal end of the endoscope taken at line III-III of FIG. 2;

FIG. 2C is a perspective view of a distal end of an alternative embodiment of the distal end of an endoscope lens protection apparatus;

FIG. 3 is a perspective view of the endoscope lens protection apparatus of FIG. 2 including a sealing mechanism;

FIG. 4 is a perspective view of the endoscope lens protection apparatus of FIG. 3 inserted into a trocar;

FIG. 5 is an exploded view of a housing member of the endoscope lens protection apparatus of FIG. 3;

FIG. 6 is a perspective view of a distal end of another embodiment of an endoscope lens protection apparatus;

FIG. 7 is a further perspective view of the endoscope lens protection apparatus distal end of FIG. 6 including an indexable film;

FIG. 8 is another perspective view of the endoscope lens protection apparatus distal end of FIG. 6 including further features;

FIG. 8A is an enlarged view taken at line IV-IV of FIG. 8;

FIG. 9 is an alternative embodiment of an endoscope lens protection apparatus;

FIG. 9A is an enlarged view taken at line V-V of FIG. 9; and

FIG. 10 is a process flow diagram summarizing an embodiment of a method of operation.

### DESCRIPTION OF THE PREFERRED EMBODIMENTS

The present invention may be further understood with reference to the following description and the appended drawings, wherein like elements are referred to with the same reference numerals. For purposes of the description hereinafter, spatial orientation terms, as used, shall relate to the referenced embodiment as it is oriented in the accompanying drawing figures or otherwise described in the following detailed description. However, it is to be understood that the embodiments described hereinafter may assume many alternative variations and configurations. It is also to be understood that the specific components, devices, features, and operational sequences illustrated in the accompanying drawing figures and described herein are simply exemplary and should not be considered as limiting.

The present invention relates to endoscopic devices and, in particular, relates to a system and method of maintaining a clear field of view through an endoscope during a minimally invasive surgery. Exemplary embodiments of the present invention provide an indexable film and an air seal, for example. Although the term endoscope is used herein throughout, it is contemplated and understood by those of skill in the art that the described and claimed systems, apparatuses and methods may be used for any device that is inserted into a human and/or animal body for optical examination by a user.

Generally, the below described embodiments ensure a clear and unobstructed field of view through an endoscope during the course of a minimally invasive surgery. A continuous strip of film or tape is incrementally indexed in front of an endoscope lens while riding upon a thin boundary layer of compressed gas, in one embodiment, for example. As the film becomes soiled, it may be incrementally advanced from within a sheath so that the blemished portion advances from view and is replaced by a portion of clean tape. A compressed gas accompanies this film through the sheath and provides a critical seal which prevents soiling of the tape's underside as it passes in front of the lens. This action instantly clears the field of view of an endoscope under the most extreme of soiling conditions.

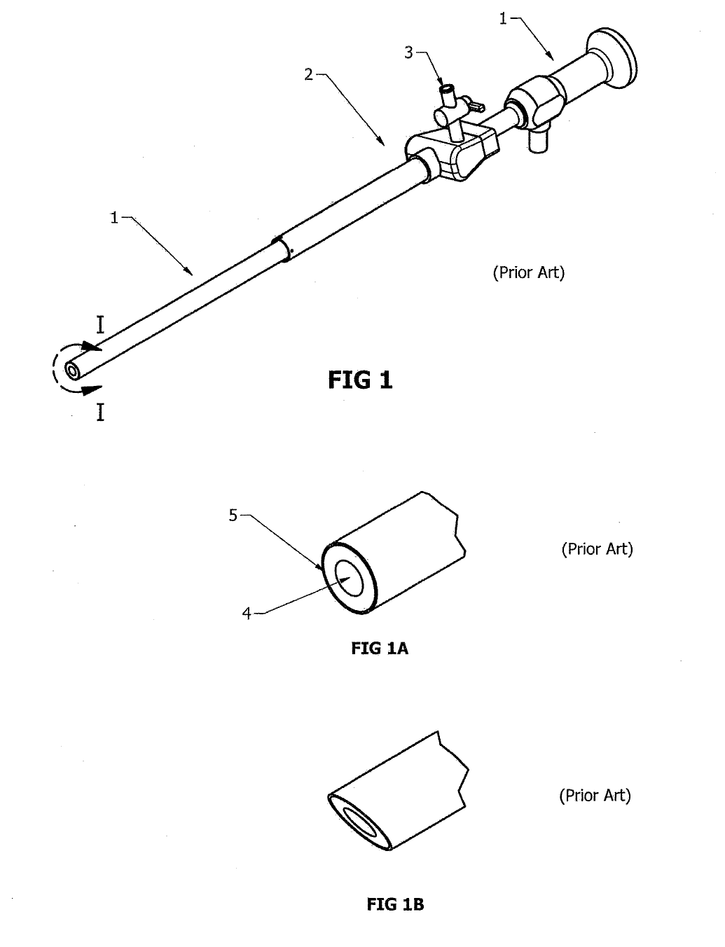

FIGS. 1-1B depicts some of the typical prior art components used during a minimally invasive surgery. A rigid endoscope 1 having an endoscope body, or shaft, having a proximal end and a distal end may be inserted through a trocar 2, which may in turn be inserted through a bodily incision. The trocar acts as a sleeve through which the endoscope accesses the bodily cavity. The trocar also has a port 3 through which insufflation gas may be introduced into the bodily cavity.

FIG. 1A highlights the scope tip, at which the viewing lens 4 and a fiber optic light emitting ring 5 may be found. FIG. 1A shows a standard endoscope tip. FIG. 1B shows a different variation of a scope in which the tip and its lens are raked at an angle, usually 30, 45, or 60 degrees. These endoscopes may also have various shaft diameters; usually ranging from 4 mm-10 mm.

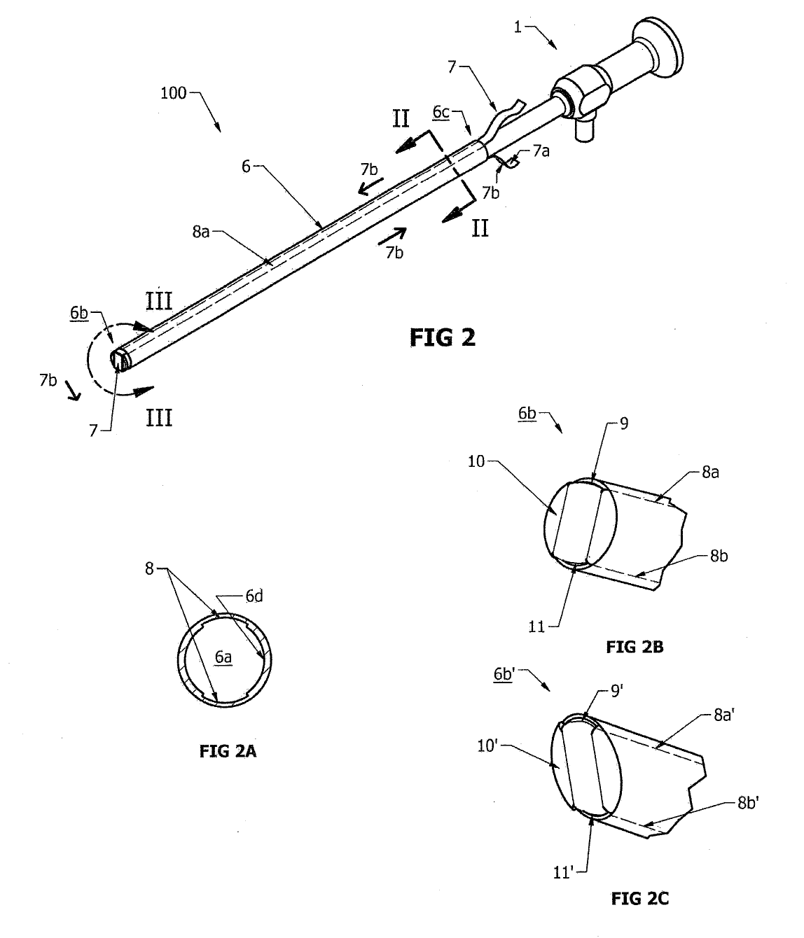

Referencing FIGS. 2 and 2A, an endoscope lens protection apparatus 100 may include sheath 6 having a proximal end and a distal end defining a chamber 6a therebetween configured to receive the body of the endoscope 1. The sheath 6 may be affixed axially over the body of the endoscope 1. A thin film or tape 7, such as a clear film 7, having an underside surface 7a and an exposed surface 7b (see FIG. 7), which permits visibility therethrough, may enter and extend through the sheath 1 at the proximal end 6c thereof, and runs axially to the distal end 6b through a clearance (see FIG. 2B) afforded between the sheath 6 and scope 1 to the distal end of the sheath 6. The interior surface 6d defines two detents 8, as shown in FIG. 2A, extend over the interior surface 6d of the sheath 6 from the proximal end 6c to the distal end thereof, as indicated by broken lines 8a in FIG. 2 (see also broken line 8a′ in FIG. 2C) thereby defining a film supply channel 8a and a film retraction channel 8b. Film retraction channel 8b is also shown by broken lines 8b, 8b′ of FIGS. 2B and 2C, respectively. These notches allow sufficient clearance between the body of the endoscope 1 and sheath 6 for the film 7 to travel between the proximal end 6c and distal end 6b thereof.

Specifically referring now to FIG. 2B, the distal end 6b or tip of the sheath 6 which defines a film supply aperture 9 and a film retraction aperture 11. A guide surface 10, which may be clear to permit viewing therethrough, is positioned at the distal end 6b of the sheath 6 and extends between the two apertures 9, 11 to support the film 7 over the lens 4. In this manner, in use, the film 7 may emerge from a clean film position, such as, film supply channel 8a of the sheath 6 through a film supply aperture 9 defined in the distal end 6b of the sheath 6 and passes in front of the endoscope lens 4, riding over and along surface 10. The film 7 then may reenter the sheath 6 through a film retraction aperture 11 into a used film position, such as film retraction channel 8b, out the endoscope field of view and return to the proximal end 6c in a similar manner; whereupon it may exit the proximal end 6c of the sheath 6. At the same time, a used portion of film 7 will be advanced through a film supply aperture 9 in front of the endoscope lens 4. The direction of film advancement is indicated by arrows 7b of FIG. 2. Surface 10 may be constructed of an optically clear material such as acrylic, so that an undistorted clear field of view is maintained though both surface 10 and the film 7. Since this surface may be optically clear, light emitted from the fiber optic ring (see FIG. 1A, 5) may also pass through it with minimal refraction.

FIG. 2C shows a sheath tip, or distal end 6b′, designed for a scope with a tip angle greater than zero degrees, such as that shown in FIG. 1B. The same features described in FIG. 2B exist here, such as a film supply aperture 9′, a film supply channel 8a′, a film retraction aperture 11′, a film retraction channel 8b′, and a guide surface 10′.

Under a mild condition of soiling, such as a splatter of blood or debris which deposits itself on the surface of the film 7, a simple advancement of the film 7 by an increment approximately equal to the diameter of the lens 4 may be sufficient. In this regard the tape alone may act as a splash guard. However, under moderate to severe soiling conditions, such as when larger amounts of liquid contaminants such as blood contact the sheath distal end 6a, a splash guard of this sort is not alone sufficient. Therefore, it may be necessary to provide some sort of sealing mechanism to prevent blood or other debris from penetrating into a position underneath the film 7 and in front of the lens 4.

As illustrated, for example, in FIG. 7, the position may be more specifically defined as the space residing between the underside surface 7a of film 7 and surface 10. Although the tape may be pulled taught along surface 10, liquid may seep underneath the film 7 through capillary forces. Once this underside surface 7a of the film 7 is reached by contaminant, any continued indexing of the film 7 may become futile, as the contamination only smears over surface 10 or lens 4 in this critical area. A sealing mechanism associated with the sheath configured to seal the underside surface of the film from debris may therefore be required to prevent liquid and other contaminants from seeping underneath the film 7 and from also backing inside the sheath chamber 6a. A sealing mechanism provides the advantage that contaminants are not allowed to seep back inside the sheath 6 through aperture 9 into film supply channel 8a, where clean film 7 awaits future use, and that contaminants are not permitted to enter aperture 9 or 11 and infiltrate the sheath chamber 6a to such a degree that the contaminants contact the lens 4 itself.

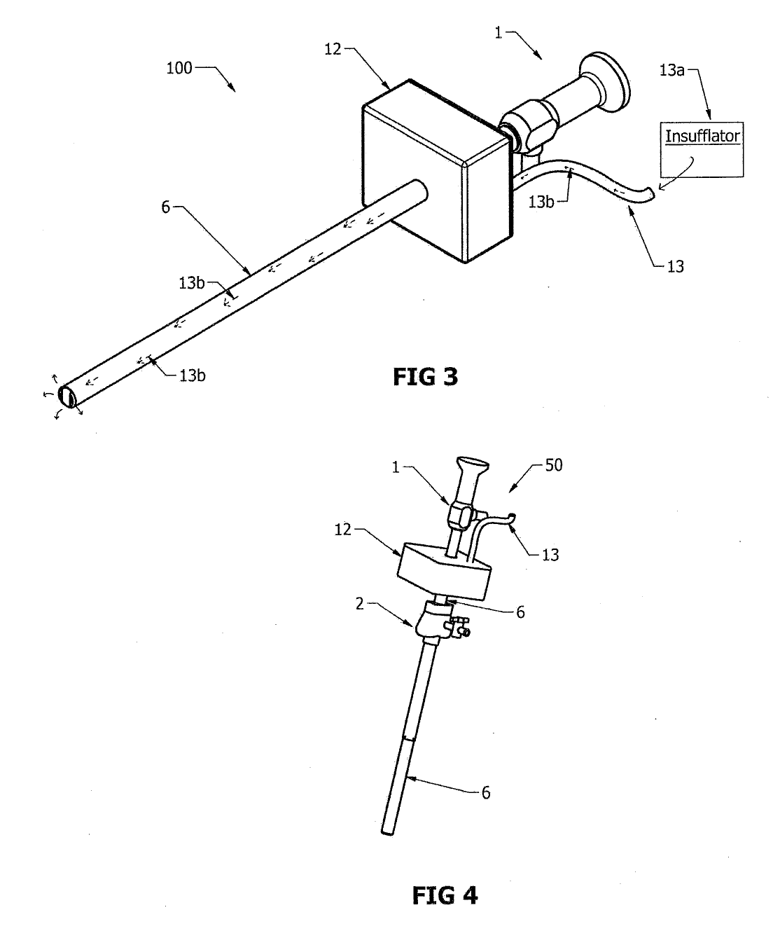

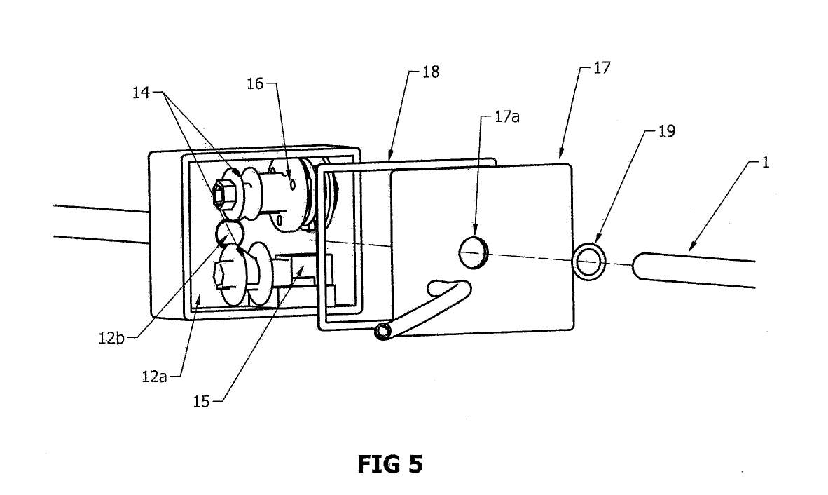

Referencing FIGS. 3 and 5, the endoscope is again shown with the sheath and tape arrangement along with an attached case housing 12. This housing 12, which defines an interior chamber 12a, serves a dual purpose. One such purpose is to provide a structure to support the film 7 supply, such as at least one clean film reservoir and at least one used film reservoir, illustrated as spools or pulleys 14 positioned in housing interior chamber 12a, and actuation components which can feed and collect film 7 into the sheath 6 in a continuous and taught manner. The other purpose is to act as an airtight chamber, i.e., housing interior chamber 12a, for which to hold a slightly compressed volume of gas relative to the ambient pressure in the operating room, as explained in more detail herein below. This housing 12 may be present, for example, as either a separate unit that is affixed to the sheath 6 in an airtight manner, or, alternatively, integrally connected with the sheath 6, such that the sheath 6 and housing 12 may comprise a unitary piece.

As illustrated in FIG. 3, the sealing mechanism may take the form of a gas-seal system configured to seal the underside surface 7a of the film 7 from debris. As shown, gas-seal system may include a gas flow line 13, which may, as illustrated, take the form of a hose, attached to and in fluid communication with the interior chamber 12a of the housing 12 and supplies a flow of compressed gas (such as, for example, Carbon Dioxide CO₂) to the housing chamber 12a. The other end of this hose is attached to a compressed gas supply source; such as an insufflator 13a, shown schematically. Although an insufflator 13a is a convenient and readily available source of this compressed gas, it should be understood that any pressure regulating source of such gas may be used.

The interior chamber 12a of housing 12 is in fluid communication with the sheath chamber 6a through an aperture 12b defined in the housing 12, such that a flow of gas may propagate through sheath chamber 6a, between the interior surface 6d of sheath 6 from the proximal end 6c to the distal end 4b of the sheath 6, for example, through film supply channel 8a and film supply channel 8b (see FIGS. 2-2B), and between the underside surface 7a of the film 7 and the surface 10 (see FIG. 7). The broken lines 13b in FIG. 3 depict the flow of the compressed gas. At the distal end 6b, the gas permeates from multiple positions both underneath and above the film 7.

Generally, in such a system, an insufflator, such as insufflator 13a, generally includes a maximum infusion pressure, for example, about 55 mm Hg. However, the flow rate of the gas-seal system, i.e., through the sheath 6 and out distal end 6b, may be relatively low, for example, 4 liters per minute, to maintain minimal risk to the patient. This is so because the pressure at an operating site, such as an abdominal cavity, is usually maintained only at 15 mm Hg. The flow should be low enough that the pressure thereof will drop to ambient environment pressure, for example in the abdominal cavity (15 mm Hg) upon emerging from distal end 6b of sheath 6. This will prevent any increase in pressure in the operating site and/or incision.

While the present embodiment incorporates a protective sheath and a receiving port for compressed gas in a manner similar to a trocar, it may not replace the requirement for a trocar. And, in fact, the sheath 6 of the present embodiment could fit inside a trocar 2 as depicted in FIG. 4. Therefore, the chosen trocar may have to accommodate the slightly increased diameter of the endoscope and sheath arrangement.

FIG. 5 shows an exploded view of the housing 12 from a rear aspect. The interior housing chamber 12a, as explained above, receives and supports the clean film and used film spools 14, which individually hold either the reservoir of clean, unused tape, or the reservoir of soiled used tape. In the illustrated embodiment, the housing chamber 12a also includes a film actuating mechanism including an electric motor 15 configured to feed and retract the film 7 through film supply channel 8a and film retraction channel 8b, and also includes a slip clutch device 16 to ensure that film 7 is continually maintained in a taught condition. At the rear of the housing assembly resides a removable back plate 17 which, in turn, is connected to gas flow line 13 providing flow through an aperture 17a defined therein. A gasket 18 serves to seal back plate 17 to a mating surface on the housing chamber. Back plate aperture 17a may be a grooved hole to accommodate an O-ring 19. When the endoscope 1 is inserted into the housing assembly the O-ring 19 is compressed and provides an airtight seal between the endoscope and the housing back plate 17.

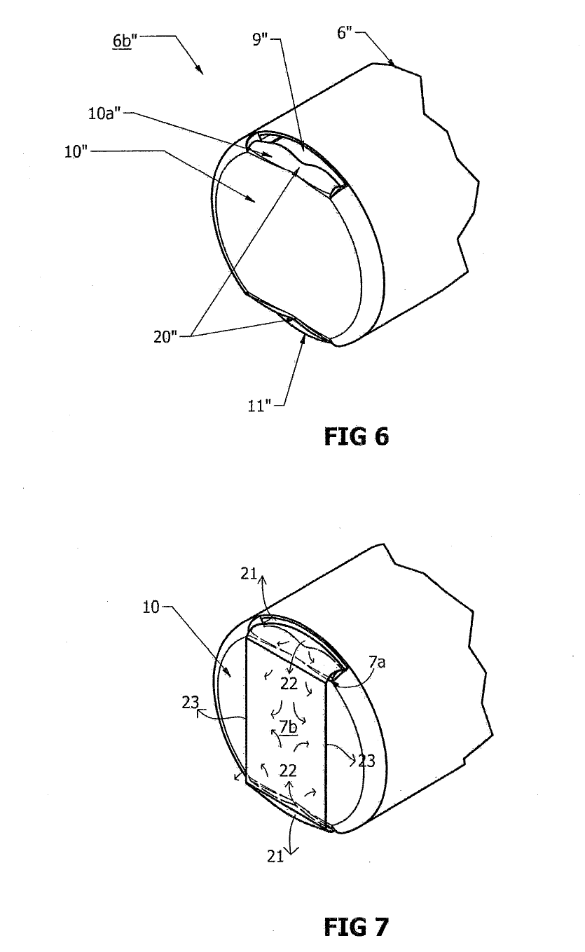

FIG. 6 details another embodiment of a sheath distal end 6b″, or sheath tip, of a sheath 6″. It should be understood that these details also exist in the sheaths with various viewing angles including a zero viewing angle, as illustrated. Surface 10″ is configured to transition the tape 7″ from an arc-like configuration, as it emerges from aperture 9″, to a flat state at the location directly in front of the endoscope lens. The film 7 may make an arc-like configuration as it travels through sheath 6″ due to the shape of the sheath and endoscope 1. As shown, surface 10″ is flat. As the arched film 7″ comes through aperture 9″, over surface 10″, because surface 10″ is flat and the film 7″ is maintained in a taught condition due to, for example, the slip clutch device 16″, the film 7″ will be transitioned from being arched to being flat over surface 10. Moreover, the sheath distal end 6b″ may define detent 20″ directly adjacent to and in communication with the film supply aperture 9″, for example directly below. As illustrated, the detent 20″ may be defined by the top edge 10a″ of surface 10″. The detent 20″ of surface 10″ may be humped or heart shaped. As shown, a similar detent 20″ may also be included to be directly adjacent to and in communication with film retraction aperture 11″. As the film 7 rides along surface 10″, detent 20″ can be considered to represent a gap between the film 7 and surface 10″. This gap forms a passageway for compressed gas to travel, as explained below.

FIG. 7 depicts the sheath distal end 6b″ with the film 7 included. Lines 21, 22, 23 indicate the flow pattern of the gas, when a gas-seal system is in use with the sheath 6 and film 7. As this air reaches the distal end 6b″ of the sheath 6″ it may take one of several paths. One such path is for the air to exit along the top of the film 7 and out of the apertures 9, 11. This path is shown by the arrows marked on lines 21. This path prohibits contamination from penetrating the interior chamber 6a″ of the sheath. Another path is for the gas to flow between the film 7 and the lens 4, or, as illustrated, guide surface 10″ through the gap or detent 20″ and is shown by arrows marked on lines 22. This phenomenon occurs symmetrically in such a manner that the opposing gas flows underneath the film 7, between the film underside surface 7a and the guide surface 10″, and causes a stagnation pressure directly in front of the lens 4. This elevated pressure causes the flow to propagate to the fringes of the tape as referenced by arrows marked on lines 23. What results is a film or boundary layer of gas between film 7 and surface 10 wherein said gas continuously emerges from the periphery of the film 7 Seepage of liquid or other contaminants into the region underneath the film 7, between the film underside surface 7a and the guide surface 10″ is thereby prevented.

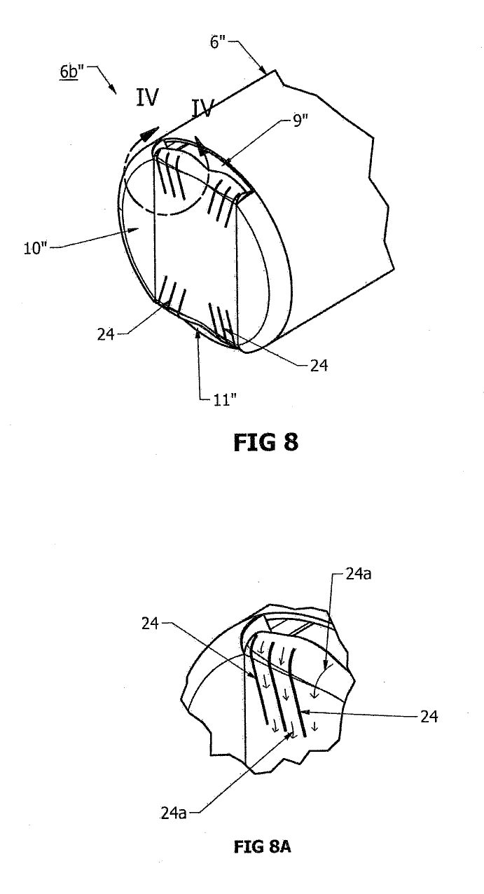

FIG. 8 shows an optional set of grooves 24 which may be defined in surface 10″ underneath film 7. As shown, the grooves 24 are defined and extend from top edge 10a″ of surface 10″ to the front of surface 10″. The grooves 24 serve to direct the pattern of gas flow underneath the tape, as indicated by lines 24a, in FIG. 8A. A tendency occurs for a larger percentage of gas to immediately dump outward to the fringes of the film in the localized regions where the film emerges and reenters the sheath 6″, such as at apertures 9″, 11″. These grooves are therefore placed in 4 localized regions as shown in FIG. 8 to mitigate this tendency and direct gas flow toward the center of surface 10″ underneath film 7. FIG. 8A shows the gas propagating parallel to the grooves 24, thus delaying this portion of gas from immediately dumping to the film edges.

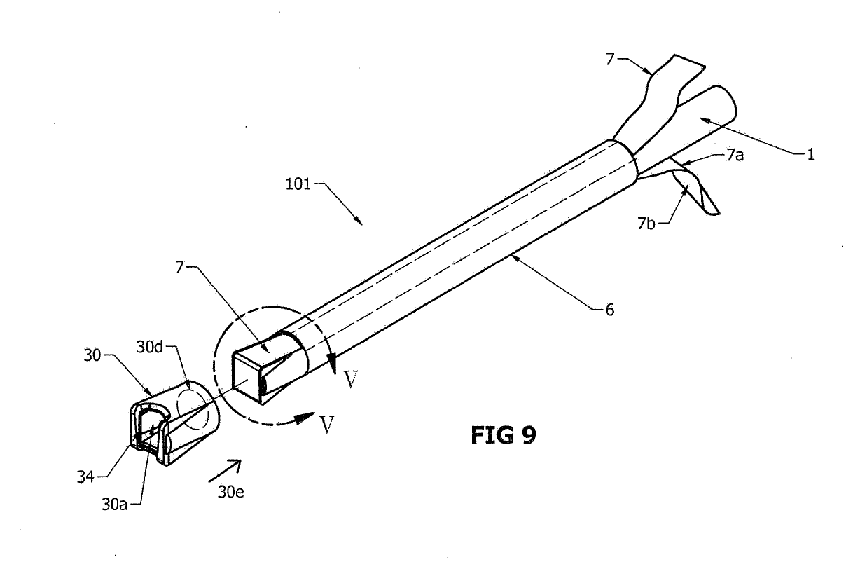

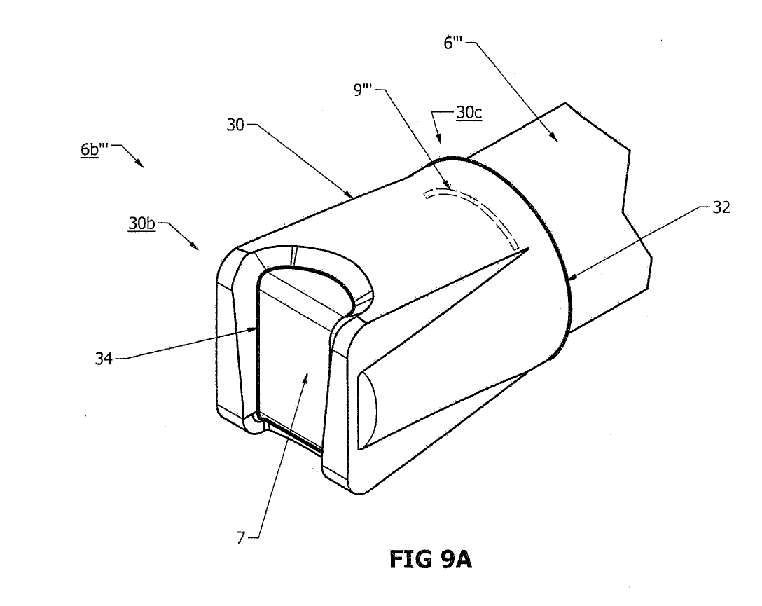

As shown in FIGS. 9-9A, an alternative embodiment of a sealing mechanism may be a boot 30 having a proximal end 30c and a distal end 30b and defining a hollow internal cavity 30a therebetween. The boot defines a proximal boot opening 30d, indicated in broken lines of FIG. 9 and a distal boot opening defined along sealing curve 17, such that the boot 30 can be positioned (see arrow 30e of FIG. 9) over distal end 6b″′ of another embodiment of the sheath 6′″, the distal end 6b″′ being received in the cavity 30a. The boot 30 may be constructed of a rubber-like or elastomeric material. This boot 30 provides a liquid tight seal in two places. One seal exists at location 32 (shown in bold) around the perimeter of the sheath 6 at an axial position proximal to film supply aperture 9″′, shown in broken lines. The other seal is a perimetric seal 34 along a closed curve shown in bold and defined by the exterior surfaces of the sheath 6″′. In this embodiment, as illustrated and can be appreciated by those skilled in the art, the aperture 9″′ may be positioned proximally of the distal end 6b″′ of the sheath 6′″. In this manner the film 7 may be fed over an exterior surface of the sheath 6 on distal end 6b″′ until it exits the distal end of the boot 30. The boot 30 pinches the film 7 against the sheath exterior surface along perimetric seal 34. The resulting sealing action prevents foreign liquid and debris from accessing the region underneath film 7 and/or into aperture 9″′ and contaminating interior sheath chamber or field of view of the endoscope 1. The seal also keeps the edges of the protective film unexposed to the environment of the bodily cavity, which prevents fluid and contaminants from seeping underneath the film 7. A sufficient tension force, for example, by electric motor 15, could be applied when advancing the film to overcome the resultant frictional force induced by the pinching action between boot 30 and film 7.

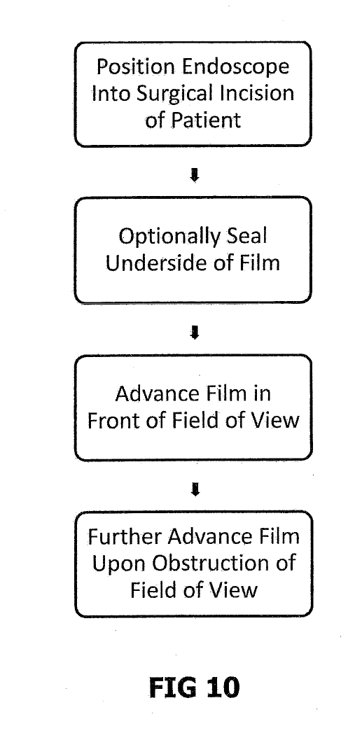

Another aspect, as summarized in the process flow diagram of FIG. 10, is a method of maintaining a clear field of view during endoscopic surgery using an endoscope 1. The method includes positioning the endoscope 1 into a surgical incision of a patient; advancing the indexable film 7 in front of the endoscope lens 4 field of view; viewing the surgery site through the endoscope lens 4 and the indexable film 7; and advancing the indexable film 7, for example, through channels 8a, 8b of sheath 6 of FIGS. 2-2B, when the endoscope field of view becomes obstructed, wherein the film positioned in the field of view is moved to a used film position, for example, through aperture 11 and into channel 8b of FIGS. 2-2B, and an unused portion of the film 7 in the endoscope field of view providing an unobstructed view of the surgery site through the endoscope lens 4. The method may also include sealing the underside surface 7a of the film 7 from debris, such as by providing a gas-flow through the sheath between an interior surface of the sheath and the endoscope body from the proximal end of endoscope to the distal end of the endoscope and between the endoscope lens and the film, as described above, or by positioning a sealing boot 30, described above defining a distal opening having a perimetric seal 34 around the distal end 6b″′ of a sheath 6″′.

While specific embodiments have been described in detail herein, it will be appreciated by those skilled in the art that various modifications and alternatives to those details could be developed in light of the overall teachings of the disclosure. Accordingly, the particular arrangements disclosed are meant to be illustrative only and not limiting as to the scope of the device of the present disclosure which is to be given the full breadth of the claims appended and any and all equivalents thereof.

The invention claimed is:

1. An endoscope lens protection apparatus, wherein the endoscope includes an endoscope body having a proximal end and a distal end and a lens positioned at a distal end of the endoscope body, the protection apparatus comprising:
a clear indexable film strip positioned over the endoscope lens having an underside surface and an opposite exposed surface, such that the underside surface is positioned to face the endoscope lens, and the incision side surface is exposed;
wherein the indexable film is movable from a clean film position to a used film position, such that, in operation, the indexable film is advanced from a point in front of the lens to a used film position out of the endoscope field of view.

2. The endoscope lens protection apparatus of claim 1, further comprising a sheath having a proximal end and a distal end defining a chamber therebetween configured to receive the endoscope body, an interior sheath surface defining a film supply channel and a film retraction channel, the supply channel and retraction channel extending from a proximal end to a distal end of the sheath, wherein the indexable film extends and is movable along the length of the film supply channel, around the point in front of the endoscope lens, and along the length of the film retraction channel.

3. The endoscope lens protection apparatus of claim 2, further comprising at least one clean film reservoir and at least one used film reservoir associated with the sheath.

4. The endoscope lens protection apparatus of claim 3, wherein the clean film reservoir comprises a spool and used film reservoir comprises a spool.

5. The endoscope lens protection apparatus of claim 4 further comprising a housing member defining an interior chamber positioned at the proximal end of the sheath, wherein the clean spool and used spool are positioned in the housing interior chamber.

6. The endoscope lens protection apparatus of claim 2, further comprising a sealing mechanism associated with the sheath, the sealing mechanism configured to seal the underside surface of the film from debris.

7. The endoscope lens protection apparatus of claim 6, wherein the sealing mechanism comprises a gas-seal system.

8. The endoscope lens protection apparatus of claim 7, wherein the gas-seal system is configured to supply a flow of gas propagating through the sheath chamber between the interior surface of the sheath and the endoscope body from the proximal end of sheath to the distal end of the sheath and between the endoscope lens and the film.

9. The endoscope lens protection apparatus of claim 8, wherein the gas-seal system further comprises a compressed air supply, the flow of gas being supplied from the compressed air supply.

10. The endoscope lens protection apparatus of claim 9, further comprising a housing member defining an interior chamber for receiving at least one clean film reservoir and at least one used film reservoir, the housing member interior chamber being in fluid communication with the compressed air supply and the sheath chamber.

11. The endoscope lens protection apparatus of claim 7, wherein the sheath comprises a film guide surface positioned at the distal end of the sheath.

12. The endoscope lens protection apparatus of claim 11, wherein the sheath distal end defines a film supply aperture and film retraction aperture and at least one detent positioned directly adjacent in communication with the film supply aperture.

13. The endoscope lens protection apparatus of claim 12, wherein the guide surface defines a plurality of groves extending from the at least one detent to the film guide surface.

14. The endoscope lens protection apparatus of claim 6, wherein the sealing mechanism comprises a sealing boot comprising a boot body having a proximal end and a distal end and defining a hollow internal cavity having a proximal boot opening and a distal boot opening, wherein the boot body is positioned over the sheath, such that the distal end of sheath and endoscope body are inserted into the boot internal cavity through the proximal boot opening, the distal boot opening including a perimetric seal configured to seal the film underside surface from debris.

15. A method of maintaining a clear field of view during endoscopic surgery using an endoscope including an endoscope body having a proximal end and a distal end and a lens positioned at a distal end of the endoscope body, the method comprising the steps of:
positioning the endoscope into a surgical incision of a patient;
advancing an indexable film in front of the endoscope lens field of view;
viewing the surgery site through the endoscope lens and the indexable film; and
advancing the indexable film, when the endoscope field of view becomes obstructed, wherein the film positioned in the field of view is moved to a used film position, and an unused portion of the film in the endoscope field of view providing an unobstructed view of the surgery site through the endoscope lens.

16. The method of claim 15, wherein the step of advancing the indexable film comprises the step of advancing the film through a sheath positioned around the endoscope body.

17. The method of claim 16, further comprising the step of sealing an underside surface of the film.

18. The method of claim 16, wherein the step of sealing comprises providing a gas-flow through the sheath between an interior surface of the sheath and the endoscope body from the proximal end of endoscope to the distal end of the endoscope and between the endoscope lens and the film.

19. The method of claim 17, wherein the step of sealing comprises positioning a sealing boot defining a distal opening having a perimetric seal around the distal end of the sheath.

20. An endoscope system comprising:
an endoscope body having a proximal end and a distal end and a lens positioned at a distal end of the endoscope body;
an indexable film strip; and
a sheath having a proximal end and a distal end defining a chamber therebetween configured to receive the endoscope body, an interior surface of the sheath defining a film supply channel and an opposite film retraction channel, the supply channel and retraction channel extending from the proximal end to the distal end of the sheath, wherein the indexable film extends from and is movable from the proximal end of the sheath, through the film supply channel, around a point in front of the endoscope lens in the endoscope field view, and through the film retraction channel back to the proximal end of the sheath.
